(19) United States
(12) Patent Application Publication
(10) Pub. No.: US 2023/0030753 A1
(43) Pub. Date: Feb. 2, 2023

(54) METHOD FOR DETECTING SHORT-CHAIN FATTY ACIDS IN BIOLOGICAL SAMPLE

(71) Applicant: National Taiwan University, Taipei (TW)

(72) Inventors: Cheng-Chih HSU, Taipei (TW); Ting-Hao KUO, Taipei (TW); Cheng-Yu WENG, Taipei (TW); Laura Min Xuan CHAI, Taipei (TW); Hsin-Bai ZOU, Taipei (TW)

(73) Assignee: National Taiwan University, Taipei (TW)

(21) Appl. No.: 17/648,618

(22) Filed: Jan. 21, 2022

(30) Foreign Application Priority Data
Aug. 2, 2021 (TW) 110128349

(51) Int. Cl. G01N 33/483; H01J 49/16; G01N 1/38; G01N 1/40; H01J 49/00

(52) U.S. Cl. CPC G01N 33/4833; H01J 49/16; G01N 1/38; G01N 1/4044; H01J 49/0031; H01J 49/004

(57) ABSTRACT

The present disclosure provides a method for detecting short-chain fatty acids in biological samples, including a derivatizing step, a loading step and a detecting step. The derivatizing step includes treating the short-chain fatty acids in the biological sample with 2-nitrophenylhydrazine for derivatizing the short-chain fatty acids into a sample to be detected. The loading step includes loading the sample onto a paper carrier. The detecting step includes analyzing the sample loaded onto the paper carrier by direct analysis in real time mass spectrometry for obtaining a detection result. The method provided by the present disclosure may complete the analysis of the biological sample within a short period of time and achieve a quantitative result comparable to that obtained by conventional chromatographic approaches.

### CROSS-REFERENCE TO RELATED PATENT APPLICATION

This application claims the benefit under 35 U.S.C. § 119 of TW Patent Application Ser. No. 110128349 filed on Aug. 2, 2021, which is incorporated herein by reference in its entirety.

Some references, which may include patents, patent applications and various publications, may be cited and discussed in the description of this disclosure. The citation and/or discussion of such references is provided merely to clarify the description of the present disclosure and are not an admission that any such references are “prior art” to the disclosure described herein. All references cited and discussed in this specification are incorporated herein by reference in their entirety and to the same extent as if each reference was individually incorporated by reference.

### FIELD OF THE DISCLOSURE

The present disclosure relates to a method for detecting short-chain fatty acids in biological samples, and more particularly to a method for detecting short-chain fatty acids in biological samples using a paper carrier.

### BACKGROUND

The influence of gut microbiota on human health has received extensive attention over the past few decades. The gut microbiota may form metabolic pathways in the host, such pathways having the ability to produce diverse metabolites that are beneficial for human health.

Among the various metabolites, short-chain fatty acids (SCFAs) are one of the kinds being most extensively discussed. SCFAs are C6-aliphatic carboxyl acids which may be produced by the fermentation of dietary fibers, and may be transported through blood circulation, such that they not only affect gastrointestinal physiology but also help provide the host's energy metabolism. The functional roles of SCFAs have been implicated in the development, prevention, and treatment of various diseases, such as inflammatory bowel disease (IBD), neuropsychiatric disorders, and kidney diseases.

Besides, the level of endogenous SCFAs provides an insight into the interplay between dietary intake, composition of gut microbiota, and host metabolism. Therefore, SCFAs have been considered as potential diagnostic biomarkers of diseases. For example, the difference between the fecal concentration of propionic acid and butyric acid has been shown to be effective in discriminating irritable bowel syndrome (IBS) patients from healthy subjects. SCFAs in feces can also be used to diagnose colorectal cancer (CRC) and adenomatous polyposis (AP). In addition, the plasma's SCFA level was used as a predictive marker to cardiovascular disease outcomes in chronic kidney disease (CKD) patients. Given these vital roles of SCFAs on clinical diagnosis, an analytical method that enables a high-throughput, robust, sensitive quantification of SCFAs is needed.

In the existing art, the quantification of SCFAs in biological samples is typically carried out with separation-based techniques, for example, gas chromatography (GC) and liquid chromatography (LC) alone or in combination with mass spectrometry (MS), such as GC-MS and LC-MS. These approaches allow the reliable quantification of SCFAs in biological samples. However, because at least several to tens of minutes of instrument time are required to complete the analysis of one single sample, its throughput is inevitably compromised, and the quantification analysis cannot be carried out efficiently in a timely manner. In addition, the properties of low molecular weight, high polarity and high volatility of SCFAs lead to the derivatization process for altering the chemical properties thereof before performing quantification by the conventional high performance liquid chromatography (HPLC). Under the above circumstances, the analysis process is time consuming, for example, often taking around 45 minutes to complete analysis of one single sample. Therefore, the conventional analysis process has the disadvantages of being time consuming, labor intensive, etc., which results in unfavorably high costs. Regarding real time analysis, the existing art has disclosed the use of ambient ionization mass spectrometry for performing quantification on biological molecules. However, based on the properties of SCFA molecules, there still exists significant difficulty in analyzing these molecules directly.

### SUMMARY

In response to the above-referenced technical inadequacies, the present disclosure provides a method for detecting SCFAs in biological samples, which can complete the analysis of a single sample within a very short period of time.

In one aspect, the present disclosure provides a method for detecting short-chain fatty acids in a biological sample, comprising: a derivatization step that includes treating the short-chain fatty acids in the biological sample with 2-nitrobenzhydrazide (2-NPH) for derivatizing the short-chain fatty acids in the biological sample into a sample to be detected; a loading step that includes loading the sample to be detected onto a paper carrier; and a detecting step that includes analyzing the sample to be detected loaded on the paper carrier by direct analysis in real time mass spectrometry (DART-MS) for obtaining a detection result.

In a preferable embodiment of the present invention, the biological sample is at least one selected from the group consisting of serum, plasma, tissue, feces, fermented product of metabolite and the derivatives thereof.

In a preferable embodiment of the present invention, before the derivatization step, the method further includes a pre-treatment step that involves extracting the biological sample by a reagent for obtaining a mixture containing the short-chain fatty acids.

In a preferable embodiment of the present invention, the derivatization step includes performing an amidation reaction between 2-NPH and the short-chain fatty acids for producing the sample to be detected.

In a preferable embodiment of the present invention, the paper carrier is filter paper.

In a preferable embodiment of the present invention, the detection time in the detecting step ranges from 0.1 to 2 minutes.

In a preferable embodiment of the present invention, the short-chain fatty acid includes at least one compound selected from the group consisting of acetic acid, propionic acid, butyric acid, succinic acid, lactic acid, valeric acid and 3-hydroxybutyric acid.

In a preferable embodiment of the present invention, the loading step further includes loading 0.5 microliter (μL) to 1 μL of the sample to be detected onto the paper carrier.

In a preferable embodiment of the present invention, the detection result is a quantification result.

In another aspect, the present invention provide a method for diagnosing chronic kidney disease, including: a sampling step including obtaining a biological sample containing at least a short-chain fatty acid from an organism; a derivatization step including treating the short-chain fatty acids in the biological sample with 2-nitrobenzhydrazide (2-NPH) for derivatizing the short-chain fatty acids into a sample to be detected; a loading step including loading the sample to be detected onto a paper carrier; a detecting step including analyzing the sample to be detected loaded onto the paper carrier by direct analysis in real time mass spectrometry (DART-MS) for obtaining a detection result; and an analyzing step including analyzing the detection result for obtaining a diagnosing result.

At least one of the major technical means of the present invention is that, in the method for detecting short-chain fatty acid in a biological sample provided by the embodiments of the present invention, a derivatization step is carried out on the short-chain fatty acid in the biological sample, which is conventionally very difficult to analyze directly, followed by using a paper carrier as the sampling substrate for the detection by a DART-MS technique selected from one of the ambient ionization mass spectrometry techniques. As a result, the sample analyzing process may be completed within a very short period of time while resulting in a quantification result that is comparable to conventional chromatography methods.

### BRIEF DESCRIPTION OF THE DRAWINGS

The present disclosure will become more fully understood from the following detailed description and accompanying drawings.

FIG. 1a shows an illustration of a step in a method provided by the present invention.

FIG. 1b shows a flow chart of the method provided by the present invention.

FIG. 2 is an ion chromatogram of different fatty acids in the biological sample according to the present invention.

FIG. 3 is a high resolution pDART-MS spectrum of different fatty acids in the biological sample according to the present invention.

FIG. 4 shows the calibration curves of six common fatty acids.

FIG. 5 is a graph of the concentrations of the SCFAs in the feces of the illness-induced rats and the control rats according to an embodiment of the present invention.

FIG. 6 is a graph of the concentration of the SCFAs in the serum of dialysis-dependent ESKD patients.

FIG. 7a is a growth curve of a strain used in an embodiment of the present invention.

FIGS. 7b to 7d are quantification of the SCFAs in the fermentation media produced by metabolically engineered cyanobacteria Synechococcus elongatus strains.

### DETAILED DESCRIPTION

The present disclosure is more particularly described in the following examples that are intended to be illustrative only because numerous modifications and variations therein will be apparent to those skilled in the art. Identical numbers in the drawings indicate identical components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles are used herein for the convenience of a reader, which shall have no influence on the scope of the present disclosure.

The terms used herein generally have their ordinary meanings in the art. In case of conflict, the present document, including any definitions given herein, will prevail. The same matter may be expressed in more than one way. Alternative language and synonyms may be used for any term(s) discussed herein, and no special significance is to be placed upon whether a term is elaborated upon or discussed herein. A recital of one or more synonyms does not exclude the use of other synonyms. The use of examples anywhere in this specification, including examples of any terms is illustrative only, and in no way limits the scope and meaning of the present disclosure or of any exemplified term. Likewise, the present disclosure is not limited to the various embodiments provided herein. Numbering terms such as “first,” “second” or “third” may be used to describe various components or the like, which are for distinguishing one component from another one only, and are not intended to, nor should they be construed to impose any substantive limitations on the components or the like.

As stated above, the present invention is aimed at providing a method for detecting SCFAs in biological samples. In the embodiments of the present invention, the term “biological sample” refers to at least one substance selected from the group consisting of serum, plasma, tissue, feces, fermented metabolites and derivatives thereof. More specifically, the biological sample may be a feces sample collected from an organism, such as rats (including rats with unpredictable chronic mild stress (USMS)), serum collected from human patients (such as those with chronic kidney disease), or fermented products produced by bacteria strains (such as metabolically engineered cyanobacteria Synechococcus elongatus PCC 7942 strains), with the possible types of samples not being limited to the foregoing examples.

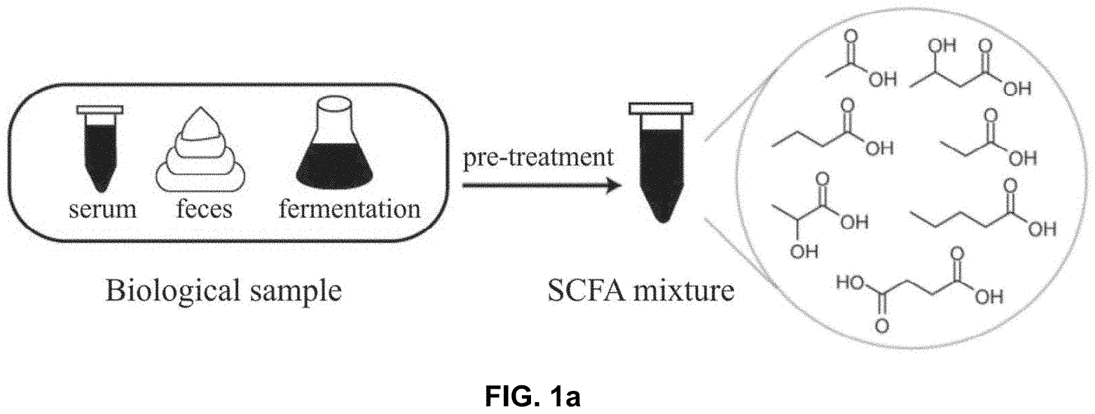

Reference is made to FIG. 1a and 1b. FIG. 1a shows an illustration of a step in a method provided by the present invention, and FIG. 1b shows a flow chart of the method provided by the present invention. As shown in FIG. 1b, the method for detecting a short-chain fatty acid in a biological sample provided by the embodiments of the present invention includes a derivatization step, a loading step and a detecting step. The derivatization step includes treating any short-chain fatty acids in the biological sample with 2-NPH for derivatizing the short-chain fatty acids into a sample to be detected.

In the present invention, the short-chain fatty acid in the biological sample includes at least one compound selected from the group consisting of acetic acid, propionic acid, butyric acid, succinic acid, lactic acid, valeric acid and 3-hydroxybutyric acid. However, the exact species of the short-chain fatty acid is not limited in the present invention.

In the embodiments of the present invention, the derivatization step includes performing an amidation reaction between 2-NPH and the short-chain fatty acid for producing the sample to be detected. In fact, the derivatization of the short-chain fatty acid by 2-NPH may enhance the effect of ionization by DART in the following steps. The various short-chain fatty acids reacted with the 2-NPH and thus amidated result in SCFA-NPH derivatives, which are products that may be detected by the analyzing instrument later. That is to say, based on the high polarity of the fatty acid, it is hard to be retained in the chromatography column and the charge-carrying effect thereof is insufficient. Therefore, the fatty acid must be derivatized to obtain a better analyzing result and to reach a desirable low detecting limit. Although constrained by the condition of the detecting instrument, the inventors of the present invention have determined that, through the derivatization of the short-chain fatty acid by 2-NPH, when compared to un-derivatized samples, the sample to be detected of the present invention may obtain a detecting signal that is up to 100 times larger.

Specifically, an internal standard (IS) solution (containing D₄-acetic acid, D₆-propionic acid and D₃-butyric acid), excess amounts of 2-NPH, pyridine and 1-ethyl-3(3-(dimethyl)propyl) carbodiimide (1-EDC) may be added to the biological sample. The biological sample may be optionally subjected to an extraction step before the derivatization step. Next, the resulting mixture (which is a yellow solution) may be spun or shaken at 950 rpm at 60° C. for 20 minutes to accelerate the reaction. The reaction may be quenched by the addition of potassium hydroxide, and the solution will turn purple if the reaction was conducted successfully. The agitation and heating process of the solution may be further conducted for another 15 minutes to ensure that the reaction is fully carried out. After cooling, phosphoric acid may be added to the solution, and the solution may be extracted with ethyl ether. The upper organic layer containing SCFA derivatives may be collected and dried by using a vacuum concentrator. The dried yellow sample was then resuspended in methanol and stored at −80° C. before analysis.

Reference is made to FIG. 1a. In some embodiments according to the present invention, before the derivatization step, the method may further include a pre-treatment step that involves extracting the biological sample with a reagent to obtain a mixture containing the short-chain fatty acid. Specifically, in some circumstances, the short-chain fatty acid in the biological sample cannot be subjected to the derivatization step directly and a pre-treatment step is required beforehand. In the embodiments of the present invention, the reagent for the pre-treatment step may be properly selected by those skilled in the art based on the type and properties of the biological sample. In a preferred embodiment, the reagent may be an organic solvent.

For example, if the biological sample is rat feces, the extraction may be conducted in a cold room at 4° C. by adding 70% methanol (water/methanol=30/70, v/v) in a ratio of 1 mL per 100 mg of feces. The sample may be then homogenized with a vortex mixer (Vortex Genie-2) for 30 minutes, followed by centrifugation at 12,500 rpm for 5 minutes. The supernatants containing SCFAs are collected and stored at −80° C. prior to the derivatization step. In another embodiment, the biological sample is a fermented product of bacteria, and a pre-treatment step may also be performed before the derivatization step. For example, for a bacterial fermentation medium, before the derivatization step, the culturing medium of S. elongatus (200 μL) may be acidified by the addition of 20 μL of hydrochloric acid (0.1 M).

Reference is made to FIG. 1B. The loading step performed after the derivatization step includes loading the sample to be detected onto a paper carrier. Specifically, the SCFAs derivatives obtained from the derivatization step, i.e., the sample to be detected, is loaded onto a carrier for performing qualitative analysis and quantitative analysis. Subsequently, in the detecting step, the sample to be detected that is loaded onto the paper carrier is analyzed by direct analysis in real time mass spectrometry (DART-MS) to obtain a detection result.

Specifically, in the present invention, the direct analysis in real time mass spectrometry using paper as a carrier is called pDART-MS (paper-loaded DART-MS). A DART SVP ion source coupled with a VAPUR interface (IonSense Inc., Sangus, USA) was used for ionization. An automatic transmission module was used as a holder to align 10 paper carriers or so for performing analysis. In a preferred embodiment of the present invention, for example, the paper carrier is filter paper, and the filter paper may be cut into a triangular shape or the like. The sample to be detected that is obtained from the derivatization step may be loaded onto the tip of the triangular filter paper, and the loading amount may be from 0.5 μL to 1 μL, while preferably around 0.75 μL. The loaded filter paper may be attached on the automatic transmission module by an attaching member, such as double-sided tape and the like.

It should be noted that by using paper as the carrier, substances having higher polarity, such as salts, in the sample may adsorbed by the carrier due to the filtering effect provided by the paper. Because a DART ionization source has a better ionization effect on non-polar substances, using a paper carrier to adsorb the substances with higher polarity may enhance the effectiveness of the DART ionization source. For example, in the existing art, a glass rod is use as the carrier, and because the glass rod merely carries the sample (with the sample attached thereon) without providing any filtering effect, the intensity of the obtained detection signal is significantly lower than that of the paper carrier adopted by the present invention.

Next, during the detection step, the mass spectrometry coupled to the DART ionization source is used to obtain the MS spectrum of the sample using negative ionization mode. In the present invention, the detection time in the detection step ranges from, for example, 0.1 minutes to 2 minutes, and preferably from 0.1 to 0.5 minutes. In fact, the number of the mass spectrometry analyzers is not limited to one. For example, two or more commercial mass spectrometry analyzers may be used in combination to analyze the samples to be detected from different types of biological samples. In the embodiments of the present invention, a set of mass spectrometer analyzers may be used to detect the SCFAs in human serum and bacterial fermentation, and another set of mass spectrometer analyzers may be used to detect the SCFAs in rat feces. For example, an Orbitrap Elite Hybrid ion trap-orbitrap mass spectrometer (Thermo Scientific, Germany) may be used for the quantification of SCFAs in human serum and in bacterial fermentation, and for high-resolution markers of the derivatized SCFAs by using tandem mass spectrometry (MS/MS). In the meantime, a SYNAPT G2-S high-definition mass spectrometer (Waters MS Technologies, Manchester, UK) may be used for the quantification of SCFAs in rat feces.

Moreover, the present invention further provides a method for diagnosing chronic kidney disease, including a sampling step, a derivatization step, a loading step, a detecting step and an analyzing step. Specifically, the method is a method for clinical diagnosis and analysis utilizing the detection method described above. Chronic kidney disease (CKD) is one of the prevalent diseases worldwide. In particular, ESKD patients require dialysis therapy (hemodialysis or peritoneal dialysis) to postpone imminent death. CKD has been known to correlate with gut microbiota dysbiosis and results in a reduction of SCFA-producing gut bacteria. Also, SCFAs have been considered as beneficial metabolites that ameliorate kidney injury. However, the physiological role of SCFAs during CKD development remains underexplored. Therefore, the present invention provides a method that adopts the pDART-MS platform described above for diagnosing CKD, which may provide advantageous effects toward the diagnosis and research of such disease. The specific example of the method is described in Example 3 below.

In the method for diagnosing chronic kidney disease provided by the present invention, the sampling step includes obtaining a biological sample including at least a short-chain fatty acid from an organism; the derivatization step includes treating the short-chain fatty acid in the biological sample with 2-NPH for derivatizing the short-chain fatty acid into a sample to be detected; the loading step includes loading the sample to be detected onto a paper carrier; the detecting step includes analyzing the sample to be detected on the paper carrier by DART-MS to obtain a detection result; and the analyzing step includes analyzing the detection result to obtain a diagnosis result.

The details of the derivatization step, the loading step and the detecting step are as those described above and are not reiterated herein. Regarding the sampling step, the specific means for carrying out such a step may be properly selected by those skilled in the art. Regarding the analyzing step, in the embodiments of the present invention, such a step may be carried out by, for example, utilizing the standard preparation of the short-chain fatty acid and depicting/utilizing the calibration curve or the like (as described later in Example 1), and the details of the analyzing process may be properly selected by those skilled in the art.

### EXAMPLE 1: QUANTIFICATION OF SCFAs

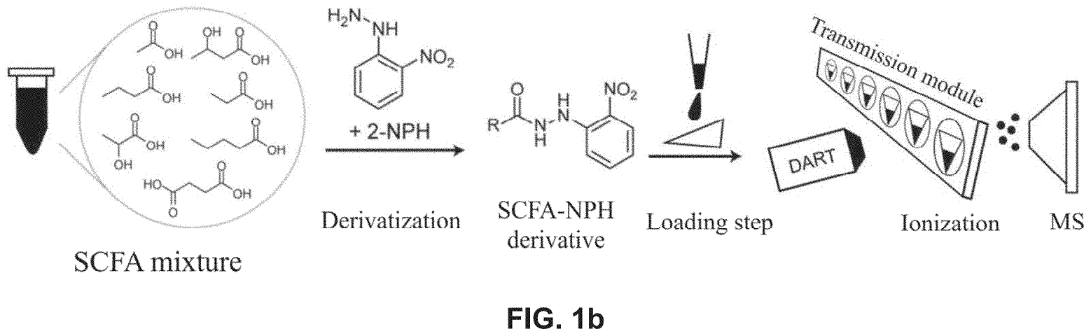

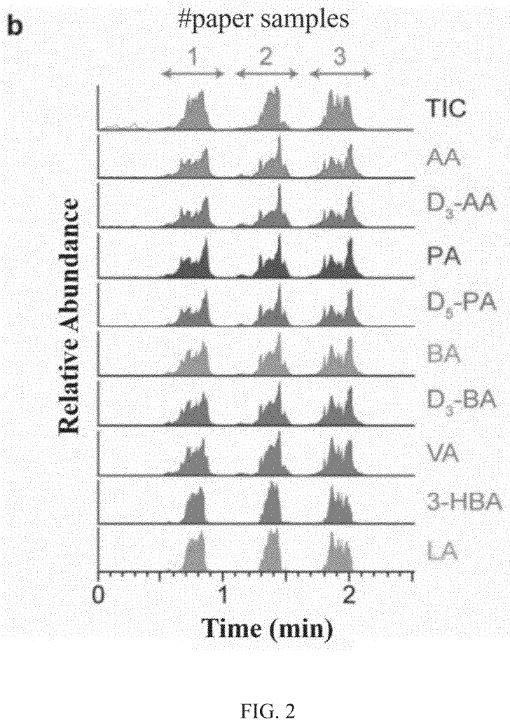

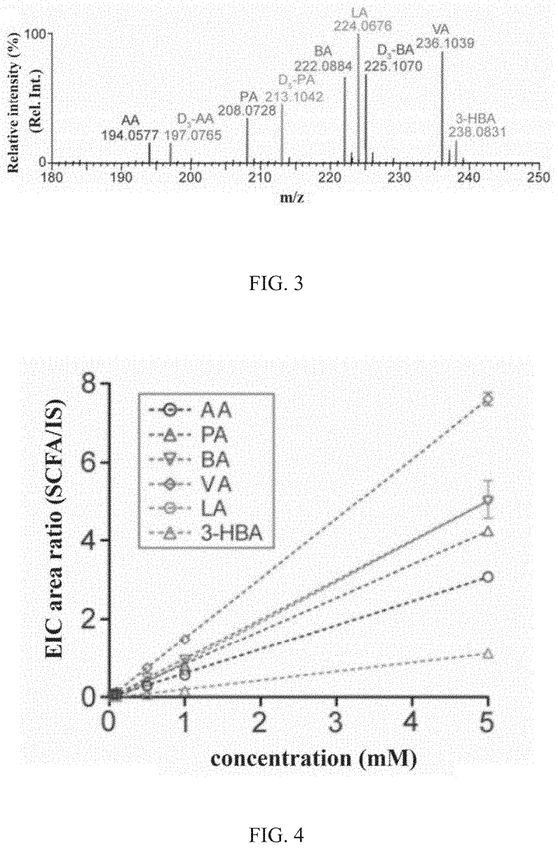

Reference is made to FIGS. 2 to 4. FIG. 2 is an ion chromatogram of different fatty acids in the biological sample according to the present invention, FIG. 3 is a high resolution pDART-MS spectrum of different fatty acids in the biological sample according to the present invention, and FIG. 4 shows the calibration curves of six common fatty acids. In the figures, AA refers to acetic acid, PA refers to Propionic acid, BA refers to Butyric acid, LA refers to lactic acid, VA refers to valeric acid, and 3-HBA refers to 3-hydroxybutyric acid.

In Example 1, a mixture of nine common SCFAs is prepared. The SCFAs includes acetic acid, propionic acid, butyric acid, valeric acid, lactic acid, 3-hydroxybutyric acid and three isotope-labeled standard preparations. As stated above, the SCFAs mixture is loaded onto three triangular pieces of paper and detected by pDART-MS. Each sample detection is completed within half a minute. As shown in FIG. 2, three continuous and repeated signals are obtained. In addition, a pDART-MS spectrum is shown in FIG. 3 and the predominant signals therein represent the deprotonated ion ([M-H]—) of the SCFA-NPH derivatives (the sample to be detected) with unobservable fragmentation. The results shown in FIG. 3 suggests that multiple SCFAs in a biological sample may be concurrently quantified with the pDART-MS platform.

Next, reference is made to FIG. 4. In order to evaluate the quantitative performance of the pDART-MS platform, the inventors of the present invention constructed the calibration curves by plotting the ion chromatogram area ratio of diagnostic ions between each SCFA and the standard preparations thereof against an SCFA concentration. In FIG. 4, the error bars represent the standard deviation calculated by technical triplicates.

Based on the results shown in FIG. 4, the method provided by the present invention enabled the quantitative measurement of multiple SCFAs in a broad linear range of concentration with good linearity and showed the limit of detection (LOD) and the limit of quantification (LOQ) at a μM level. Accordingly, it is demonstrated that the quantification detection performed according to the method provided by the present invention is a reliable process for detecting SCFAs from various biological sources.

### EXAMPLE 2: QUANTIFICATION OF SCFAs IN RAT FECES

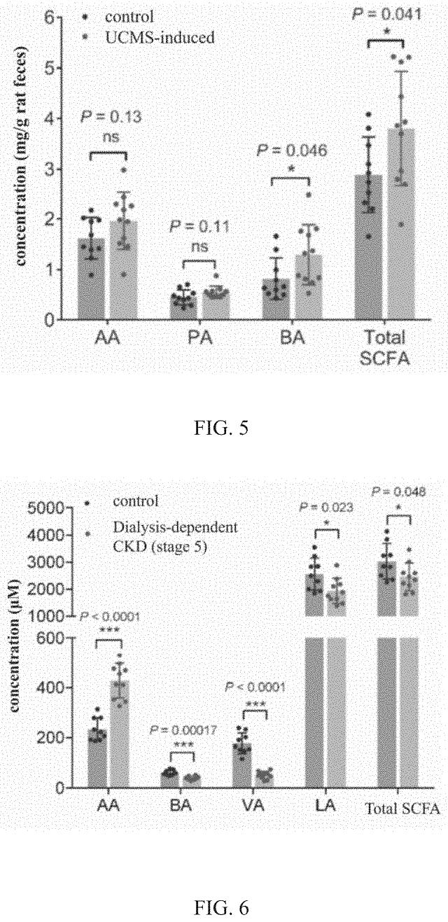

In Example 2, the SCFAs in the feces of stressed rats are quantified by the method provided by the present invention. Reference is made to FIG. 5. FIG. 5 is a graph of the concentrations of the SCFAs in the feces of the illness-induced and control rats according to an embodiment of the present invention. The total SCFA in FIG. 5 refers to a summed concentration of AA, PA and BA. Statistical significance was evaluated by an unpaired Student's t test (*P value<0.05). The error bars represent a standard deviation.

First, in Example 2, the inventors of the present invention quantified the SCFAs in 21 rat feces samples through the pDART-MS platform. The number of rats in the control group and the unpredictable chronic mild stress (USMS) group are 10 and 11, respectively. Next, the pre-treatment step and the derivatization step are performed on the rat feces as described above, and the loading step and detecting step are performed on the sample to be detected as described above. As shown in FIG. 5, three SCFAs, including acetic acid, propionic acid and butyric acid are readily detected and quantified, and the result shows a higher level of acetic acid (about 2 mg/g feces) and lower concentrations of propionic acid and butyric acid. Such an experimental result is consistent with the following study: Kelly, J. R.; Borre, Y.; O'Brien, C.; Patterson, E.; El Aidy, S.;Deane, J.; Kennedy, P. J.; Beers, S.; Scott, K.; Moloney, G.; Hoban, A. E.; Scott, L.; Fitzgerald, P.; Ross, P.; Stanton, C.; Clarke, G.; Cryan, J. F.; Dinan, T. G. J. Psychiatr. Res. 2016, 82, 109-118.

Comparing the experiment results of the two groups of rats, the inventors of the present invention determined that the butyric acid concentration in the control group was 0.82±0.41 mg/g, and the butyric acid concentration in the UCMS-induced group was 1.29±0.60 mg/g; and that the total SCFAs in the control group was 2.88±0.76 mg/g, and that the total SCFAs in the UCMS-induced group was 3.80±1.13 mg/g (i.e., the total SCFAs were slightly elevated in the UCMS-induced group). However, there was no significant difference in acetic acid and propionic acid levels between rats in the control group and the UCMS-induced groups.

In order to confirm the stability, reliability and effectiveness of the method provided by the present invention, a conventional HPLC method was also implemented to quantify SCFAs of the same batch of fecal samples on the same day, and the highly consistent results obtained suggest that the pDART-MS platform adopted according to the present invention enables a robust quantification of fecal SCFAs.

More importantly, due to its chromatography-free nature, such a pDART-MS strategy is highly competitive with the conventional methods, such as LC-MS, in terms of time consumption. That is, for 21 rat fecal samples in this example, the sample preparation including the pre-treatment step and the derivatization step required about 4 hours for both pDART-MS and HPLC approaches, while using the instrument to perform detection during the detecting step, pDART MS potentially saved dozens of hours with respect to the instrument time.

### EXAMPLE 3: QUANTIFICATION OF SCFAs IN SERUM OF CKD PATIENTS

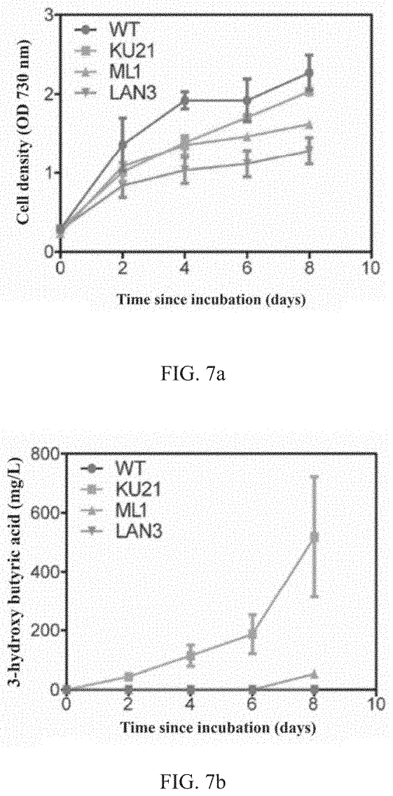

In Example 3, the pDART-MS detection method provided by the present invention is implemented to quantify the SCFAs in the serum of dialysis-dependent CKD patients. Reference is made to FIG. 6. FIG. 6 is a graph of the SCFAs concentration in the serum of dialysis-dependent ESKD patients. Four SCFAs in the serum, including acetic acid, butyric acid, valeric acid, and lactic acid were measured in both dialysis dependent CKD patients and healthy controls. Specifically, the SCFAs in the serum were dominated by lactic acid (about 2000 μM), whereas the levels of the other SCFAs varied from 40 to 400 μM. Such results are similar to previous reports.

Moreover, in Example 3, compared to the healthy controls, dialysis-dependent CKD patients exhibited twice as high a level of acetic acid in their serums but significantly decreased levels of butyric acid, valeric acid (including straight-chain and branched-chain isomers), lactic acid, and total SCFA in the serums. However, no significant differences in the levels of SCFAs in the serums were noted between stage 5 nondialysis-dependent CKD patients and healthy individuals.

As mentioned above, the physiological role of SCFAs during CKD development remains underexplored. Wang et al. (Wang, S.; Lv, D.; Jiang, S.; Jiang, J.; Liang, M.; Hou, F.; Chen, Y. Clin. Sci. 2019, 133, 1857-1870) have compared SCFA levels in the serum of CKD patients at different stages with that of healthy individuals and suggested that SCFAs in the serum may be used as potential biomarkers for the assessment of kidney functions. In this study, the researchers demonstrated a reduction of butyric acid in the serum of the CKD patients of all stages, while identifying a slight reduction of acetic acid in the serum of the stages 1-4 CKD patients but not in the stage 5 patients, when compared with healthy individuals. Similar to their findings according to the results of Example 3, the inventors of the present invention identified a reduction of butyric acid, valeric acid, and lactic acid in the serums of dialysis dependent CKD patients, which possibly resulted from the removal of SCFAs in the serums by hemodialysis.

In contrast, in Example 3, the acetic acid levels in the serum of the dialysis-dependent CKD patients were elevated, which is contradictory to the aforementioned study. Acetic acid is well known as an end product of gut microbial fermentation, but in fact, its circulating level is concurrently dependent on folate-dependent acetyl-CoA hydrolysis, a biochemical event in which arylamine N-acetyltransferases (NATs) hydrolyze acetyl-CoA to release acetic acid in the presence of folate. Therefore, the inventors of the present invention postulate that such an NAT-dependent pathway may lead to an increase in acetic acid in the serum of dialysis-dependent CKD patients because of regular folate supplements following the recommended guidelines for controlling this disease. As a result, the method provided by the present invention may be used to evaluate the change of concentration of the SCFAs in serum as potential biomarkers in the CKD patients.

### EXAMPLE 4: QUANTIFICATION OF SCFAs PRODUCED BY MICROBIAL CELL factories

In Example 4, the SCFAs produced by microbial cell factories directly from fermentation media are detected. In fact, biochemical production through engineered microorganisms is considered to be a next-generation green chemical production method. In Example 4, the culture media of photoautotrophic cyanobacterium Synechococcus elongatus PCC 7942 strains, including ML1, KU21 and LAN3 were examined. These S. elongatus strains have been metabolically engineered to efficiently produce specific SCFAs via photosynthesis. Specifically, strains ML1, KU21, and LAN3 are able to produce butyric acid, 3-hydroxybutyric acid, and succinic acid, respectively, by using carbon dioxide under sunlight. These strains were cultured herein, the culture media thereof were harvested every other day, and the SCFA production was quantified with pDART-MS platform adopted by the present invention.

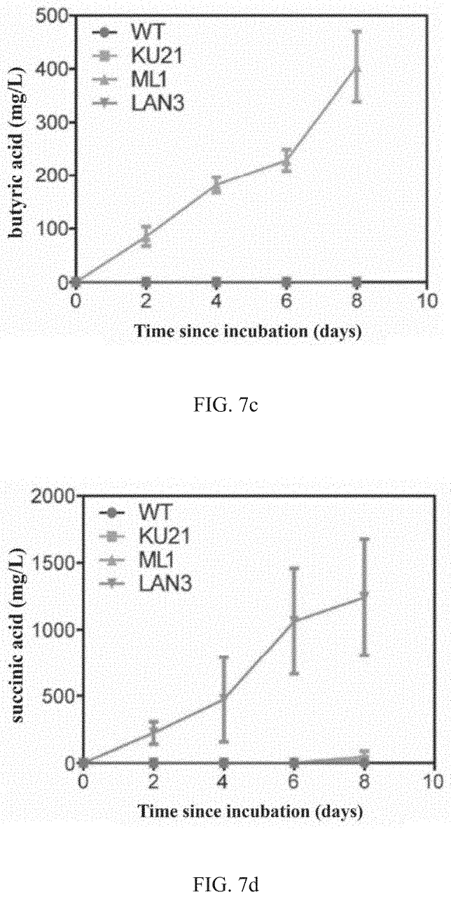

Reference is made to FIGS. 7a to 7d. FIG. 7a shows the growth curve of the strains used in an example of the present invention, including a wild-type (WT) strain and three metabolically engineered strains; and

FIGS. 7b to 7d exhibit the varied concentration of the fermented SCFAs produced by the metabolically engineered S. elongatus strains. In Example 4, the production of SCFA was able to be probed as early as the following day of postinoculation. Furthermore, the accumulated amounts of SCFAs associated with the continuously growing bacteria were quantified and showed excellent consistency with the previous studies. Therefore, as demonstrated in Example 4, the method provided by the present invention has profound potential to be incorporated into the analytical pipelines of the chemical engineering industry.

In summary, one of the major technical means of the present invention is that the method for detecting short-chain fatty acid in biological samples includes performing derivatization towards the short-chain fatty acid in biological samples that are conventionally difficult to analyze directly, and then using paper as the sample carrier for detecting the short-chain fatty acid by a real time mass spectrometry (DART-MS) technique selected from one of the ambient ionization mass spectrometry techniques, and therefore, the sample analyzing process may be completed in a very short period of time while achieving quantification results consistent with the results of conventional chromatography.

Specifically, the method developed by the inventors of the present invention utilizes the pDART-MS platform for quantifying the short-chain fatty acid in biological sample under ambient conditions. Such a method may perform quantification detection for multiple short-chain fatty acids concurrently, and the instrument time is quite short (less than one minute). Therefore, compared to the separation-based methods in the existing art, the method provided by the present invention demonstrates higher efficiency when it comes to large scale quantification of biological samples.

Moreover, the method provided by the present invention conducts a mild derivatization of SCFAs in the biological samples with cost-effective chemical reagents and then utilizes commercial equipment for detection without needing any modifications to the equipment. Therefore, such a method is readily adoptable by institutional MS laboratories worldwide.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application, so as to enable others skilled in the art to utilize the disclosure and various embodiments with possible modifications as are suited to the particular use contemplated. Alternative embodiments will become apparent to those skilled in the art to which the present disclosure pertains without departing from its spirit and scope.

What is claimed is:

1. A method for detecting a short-chain fatty acid in biological sample, including:
a derivatization step, including treating the short-chain fatty acid in the biological sample with 2-nitrobenzhydrazid (2-NPH) for derivatizing the short-chain fatty acid into a sample to be detected;
a loading step, including loading the sample to be detected onto a paper carrier; and
a detecting step, including analyzing the sample to be detected loaded onto the paper carrier by real time mass spectrometry (DART-MS) for obtaining a detection result.

2. The method of claim 1, wherein the biological sample is at least one selected from the group consisting of serum, plasma, tissue, feces, fermented product of metabolite and the derivatives thereof.

3. The method of claim 1, further including, before the derivatization step, a pre-treatment step including extracting the biological sample by a reagent for obtaining a mixture containing the short-chain fatty acid.

4. The method of claim 1, wherein the derivatization step includes performing an amidation reaction between the 2-NPH and the short-chain fatty acid for producing the sample to be detected.

5. The method of claim 1, wherein the paper carrier is filter paper.

6. The method of claim 1, wherein the detection time of the detecting step ranges from 0.1 minutes to 2 minutes.

7. The method of claim 1, wherein the short-chain fatty acid includes at least one compound selected from the group consisting of acetic acid, propionic acid, butyric acid, succinic acid, lactic acid, valeric acid and 3-hydroxybutyric acid.

8. The method of claim 1, wherein the loading step further includes loading 0.5 μL to 1 μL of the sample to be detected onto the paper carrier.

9. The method of claim 1, wherein the detection result is a quantification result.

10. A method for diagnosing chronic kidney disease, including:
a sampling step, including obtaining a biological sample including at least a short-chain fatty acid from an organism;
a derivatization step, including treating the short-chain fatty acid in the biological sample with 2-nitrobenzhydrazide (2-NPH) for derivatizing the short-chain fatty acid into a sample to be detected;
a loading step, including loading the sample to be detected onto a paper carrier;
a detecting step, including analyzing the sample to be detected loaded onto the paper carrier by real time mass spectrometry (DART-MS) for obtaining a detection result; and
an analyzing step, including analyzing the detection result for obtaining a diagnosing result.
